(19) United States
(12) Patent Application Publication
(10) Pub. No.: US 2021/0196572 A1
(43) Pub. Date: Jul. 1, 2021

(54) PRODUCING EROSION-CONTROLLED RELEASE DEVICES

(71) Applicant: HEWLETT-PACKARD DEVELOPMENT COMPANY, L.P., Spring, TX (US)

(72) Inventors: Wei HUANG, Palo Alto, CA (US); Gary J. DISPOTO, Palo Alto, CA (US)

(73) Assignee: HEWLETT-PACKARD DEVELOPMENT COMPANY, L.P., Houston, TX (US)

(21) Appl. No.: 16/076,130

(22) PCT Filed: Apr. 28, 2017
(86) PCT No.: PCT/US2017/030208

§ 371 Date: Aug. 7, 2018

(51) Int. Cl. A61J 3/06; A61K 9/20; A61K 9/50; A61K 45/06; B29C 64/165; B29C 64/393; B33Y 10/00; B33Y 50/02; B33Y 80/00

(52) U.S. Cl. CPC A61J 3/06; A61K 9/2072; A61K 9/5084; A61K 45/06; A61J 2200/20; B29C 64/393; B33Y 10/00; B33Y 50/02; B33Y 80/00; B29C 64/165

(57) ABSTRACT

In an example implementation, a method of producing an erosion-controlled release device includes accessing erosion-controlled release input data, and translating the input data into an erosion-controlled release print file. The method includes executing the erosion-controlled release print file to control a 3D printing system to produce an erosion-controlled release device that comprises an active ingredient release profile based on the erosion-controlled release input data.

### BACKGROUND

Accurate delivery of ingredients such as drugs and nutrients within a user's body can improve the therapeutic and nutritional impact of such ingredients. Accurate delivery of such ingredients can involve, for example, delivering multiple different ingredients, delivering the ingredients over a desired period of time, delivering the ingredients in particular doses, delivering the ingredients in varying doses over time, and so on. Products that enable such accurate delivery can provide improved convenience for users and help to reduce overall costs for consumers by improving the effectiveness and safety of the ingredients. Such products can include, for example, pills or tablets to be ingested by a user, and implant devices to be placed on or within a particular location of a user's body.

### BRIEF DESCRIPTION OF THE DRAWINGS

Examples will now be described with reference to the accompanying drawings, in which:

FIG. 1 shows a cross-sectional view of an example 3D printing process in which erosion-controlled release devices can be produced for erosion-controlled release of active ingredients;

FIG. 2 shows a cross-sectional view of an example erosion-controlled release and a graphical illustration of the erosion-controlled release;

FIG. 3 shows cross-sectional views of additional examples of erosion-controlled release devices in which the release of active ingredients can be controlled according to the geometry of the devices;

FIG. 4 shows examples of erosion-controlled release devices exhibiting different erosion schemes;

FIG. 5a shows a perspective view of an example of a 3D printing system suitable for printing erosion-controlled release devices;

FIG. 5b shows an example of data components within erosion-controlled release input data;

FIG. 5c shows example print file components within an erosion-controlled release print file;

FIG. 6 shows a perspective view of an example of the 3D printing system in which example erosion-controlled release devices have been printed;

FIGS. 7, 8, and 9 are flow diagrams showing example methods of producing erosion-controlled release devices.

Throughout the drawings, identical reference numbers designate similar, but not necessarily identical, elements.

### DETAILED DESCRIPTION

The diagnosis of medical conditions, illnesses, general health and fitness issues, and so on, can often lead to a one-size-fits-all approach to managing such conditions, illnesses, and issues. That is, similar diagnoses often lead to the same prescribed treatments and medications. However, while a certain condition or set of conditions may be associated with a particular diagnosis, there are many factors that should be considered when determining a plan for treating such conditions. Taking such factors into account can help achieve a more effective personalized treatment. Factors that can help determine more effective personalized treatments include, for example, biological differences between different individuals such as height, weight, age, and sex; differences in the living and working environments of different individuals; and, differences in lifestyles that may impact interactions with different treatments, such as how an individual's diet may interact with a particular drug or medicine being considered for treatment.

Providing effective treatments tailored to an individual's personal physical makeup, environment, lifestyle, and so on, often involves customizing an active ingredient consumption regimen that can deliver active pharmaceuticals (e.g., drugs) and other ingredients (e.g., nutritional supplements) in varying dosages, over varying time frames, and to varying physical locations throughout the body. Thus, a doctor may prescribe drugs in a manner to try and achieve a particular therapeutic drug level within the body, such as having a constant drug concentration level within the body. However, achieving such levels using drugs that are not formulated for a controlled release may not be possible. For example, instead of achieving a constant drug concentration level within the body, the result may be an initial concentrated burst of the drug, followed by a gradual decrease in drug concentration over time. The same notion may generally apply as well when multiple drugs are involved. For example, a doctor may prescribe multiple drugs to be taken at different times and in different concentrations in order to achieve particular therapeutic levels within the body for each of the drugs. Again, achieving such levels may not be possible using drugs not formulated to provide controlled release.

As used herein, the phrase “active ingredient”, is generally intended to refer to any of a variety of active pharmaceutical ingredients, drugs, medications, nutrients, pH level modifiers, flavors, and/or other ingredients to be consumed or applied for the treatment of various medical, nutritional, and/or other health related conditions. These terms and phrases may be used interchangeably throughout this description. In addition, throughout this description “active ingredient” may be referred to in shorthand as simply “AI”.

Products have been developed to assist individuals in self-administering active ingredient treatment regimens. These products can include ingredient delivery devices such as tablets, pills, capsules, and implantable devices that provide mechanisms to enable modified release of active ingredients. Modified release of an active ingredient generally refers to a modification in how the active ingredient is to be released and absorbed into the bloodstream or surrounding tissue. By contrast, immediate release can refer to the release of an active ingredient all at once, in a single dose. Ingredient delivery devices that provide for the modified release of active ingredients can function using a variety of different delivery modes including, for example, the delivery of multiple different ingredients, the delivery of ingredients over a desired period of time, delivering the ingredients in particular doses, delivering the ingredients in varying doses over time, and so on. Thus, ingredient delivery devices can be designed to provide customized release profiles for temporal and dose controlled delivery of multiple active ingredients that are specifically tailored to the conditions and health factors of each individual.

Customizable ingredient delivery devices can help to alleviate the difficulties associated with keeping track of medications, timing medications, and taking the proper dosages of medications. Prior methods for producing such devices include, for example, tablet press machines, powder mixers, pharmaceutical milling machines, and granulation machines that enable the production of tablets in customizable sizes, shapes, colors, coatings, and so on. More recent methods for producing such devices include 3D printing methods that can provide greater customizations such as personalized drug dosing and complex drug release profiles. In some examples, 3D printing methods used for producing drug tablets can involve the use of liquid binders applied to powder-based substrates. In some cases, tablets produced by such methods can result in tablets having poor mechanical durability, poor control of release profiles, and so on. In some examples, such anomalies may be attributable to the process steps in the liquid binder-based 3D printing method.

Accordingly, some example methods described herein enable the production of ingredient delivery devices that provide for the erosion-controlled release of active ingredients (AI), such as pharmaceuticals, nutritional supplements, colorants, flavors, smells, and so on. An example workflow is described herein for the preparation of erosion-controlled release printing files for use in a 3D (three dimensional) printing process. An example 3D printing process implementing such erosion-controlled release printing files can perform layer-by-layer additive manufacturing to construct ingredient delivery devices such as tablets, pills, capsules, and implantable devices that provide erosion-controlled release profiles. Erosion-controlled release profiles can be customized to particular active ingredients as well as to particular characteristics of a user, such as a user's biological, environmental, and lifestyle factors.

In an example 3D printing process, erosion-controlled ingredient delivery devices can be built up layer-by-layer through the selective deposition (e.g., jetting) of liquid solutions and application of fusing energy to successive layers of powder material. The liquid solutions can comprise fusing agents, detailing agents, inks, and other liquids that are jettable from an inkjet printhead. The liquid solutions can also comprise an active ingredient, or multiple active ingredients. For example, jettable liquid solutions can comprise a mixture of fusing agent and an active ingredient where the active ingredient comprises a solute and the fusing agent comprises a solvent. Fusing energy can be controllably applied to each powder layer to cause selective fusing and/or sintering of the powder material in areas where the fusing agent has been applied, while areas where detailing agents have been applied can inhibit fusing and/or sintering. The controlled deposition of fusing agents, active ingredients, and/or “fusing agent—active ingredient” mixed solution (FA-AI solution) along with applications of fusing energy onto powder layers can produce an ingredient delivery device that achieves a designed erosion release profile for the active ingredient upon ingestion of the ingredient delivery device by a user. The erosion release profile can include, for example, the timing of release of an active ingredient and the dosage of active ingredient being released. In an example 3D printing process, a number of factors can be controlled and adjusted to vary both the timing and dosing of an active ingredient including, for example, the concentration of active ingredient within the fusing agent solution, the deposition pattern of the solution, and the controlled application of fusing energy to the powder layer.

In an example process, ink and other jettable liquids can function as an active ingredient transporter as well as functioning as fusing and detailing agents. In addition, in some examples jettable liquid active ingredients can also function as fusing agents. In an example process, biocompatible powder can serve as the material of the active ingredient carrier (excipient) as well as the controller of the active ingredient release profile. In some examples, erosion-controlled release profiles can be controlled in a variety of ways, including the distribution of fusing agent droplets that comprise active ingredients, the geometry of the erosion-controlled ingredient delivery device being printed (e.g., a drug tablet), the release properties of the solid powder material, the microstructure of the material and the ingredient delivery device, and so on. In some examples, active ingredients can be carried in the powder material as well as in the ink, or instead of in the ink.

In an example workflow, erosion-controlled release 3D printing files can be prepared for use in an example 3D printing process. The 3D printing files can be formatted, for example, in 3 MF (3D manufacturing format). The 3 MF is a data file format based on XML that enables the inclusion of all 3D model data such as materials, properties, and colors information within a single file. An erosion-controlled release 3D printing file can be prepared and implemented in an example 3D printing process. The erosion-controlled release 3D printing file can guide or control a 3D printing system and process to produce an erosion-controlled release ingredient delivery device such that the device exhibits a particular erosion-controlled release profile, for example, upon ingestion into an aqueous environment. Erosion-controlled release is a type of release control method for an active ingredient (e.g., a drug) where an erosion-controlled release device such as a tablet or an implant is designed to be eroded or degraded for time-controlled release of the active ingredient. The design of the erosion-controlled release device also enables dose-controlled release of the active ingredient.

An example workflow for preparing an erosion-controlled release printing file can include determining or accumulating a range of input data pertaining to both the active ingredients and the intended target (e.g., the user) of the active ingredients. An example workflow can then translate the data into an erosion-controlled release printing file that includes information, instructions, and/or commands, that an example 3D printing system can understand, interpret, and implement. An erosion-controlled release printing file can be implemented or executed by a 3D printing system to produce an erosion-controlled release device that has a particular erosion release contour that controls how the device erodes when ingested. The erosion contour enables the erosion-controlled release device to release active ingredients according to a release profile determined from the erosion-controlled release printing file.

Data determined and/or utilized as input data in an example workflow to prepare an erosion-controlled release printing file can include designed release profile data for an active ingredient. Some active ingredients can have designed or suggested release profiles that specify optimum timing and dosing to achieve the maximum therapeutic benefits from the active ingredients. Other data utilized as input data in an example workflow can include, for example, properties of the excipient powder material such as its material erosion rate according to different microstructures within an erosion environment, the concentrations of active ingredients within the inks (i.e., fusing agents) that will be deposited onto the excipient powder material in the 3D printing process, data indicating the energy absorptivity of fusing agents, data indicating an amount of cooling caused by cooling agents, hardware specification data characterizing a fusing energy source of the 3D printing device, data indicating which microstructures are to be produced within an erosion-controlled release device, geometric data indicating an external boundary and initial surface area of an erosion-controlled release device, personal data about an intended user, identification data to identify active ingredients within an erosion-controlled release device and the intended user of the erosion-controlled release device, and so on. Personal data about an intended user can include, for example, a user's height, weight, age, sex, living environment, working environment, diet, exercise habits, and so on. and other data.

With this input data, an example workflow can prepare an erosion-controlled release printing file, which can include control information such as 3D printing instructions and commands that an example 3D printing system can understand, interpret, and execute to produce an erosion-controlled release device with an erosion-controlled release profile. Information and/or instructions in an erosion-controlled release printing file can include, for example, distribution patterns that define active ingredient droplet depositions throughout the geometry of an erosion-controlled release device, the density of active ingredient droplets to be deposited throughout the geometry of an erosion-controlled release device, fusing process data including fusing intensity levels, fusing application durations, the number of fusing exposures to be applied, and so on.

In a particular example, a method of producing an erosion-controlled release device includes accessing erosion-controlled release input data, and translating the input data into an erosion-controlled release print file. The method includes executing the erosion-controlled release print file to control a 3D printing system to produce an erosion-controlled release device that comprises an active ingredient release profile based on the erosion-controlled release input data.

In another particular example, a 3D printing system for producing erosion-controlled release devices includes a memory device storing erosion-controlled release input data. The system includes a processor programmed with instructions from a print file preparation module to prepare an erosion-controlled release print file based on the erosion-controlled release input data. The print file comprises commands to control the 3D printing system to produce an erosion-controlled release device.

In another example, a non-transitory machine-readable storage medium stores instructions that when executed by a processor of a 3D printer for producing an erosion-controlled release device, cause the 3D printer to retrieve data from an erosion-controlled release input data source, and determine a release profile of an active ingredient from the data. The instructions further cause the 3D printer to determine a microstructure and a porosity of the microstructure that can achieve the release profile in an erosion environment, determine a fusing energy to sinter layers of powder material to form the microstructure with the porosity, and perform a fusing process to apply the fusing energy to the layers of powder material.

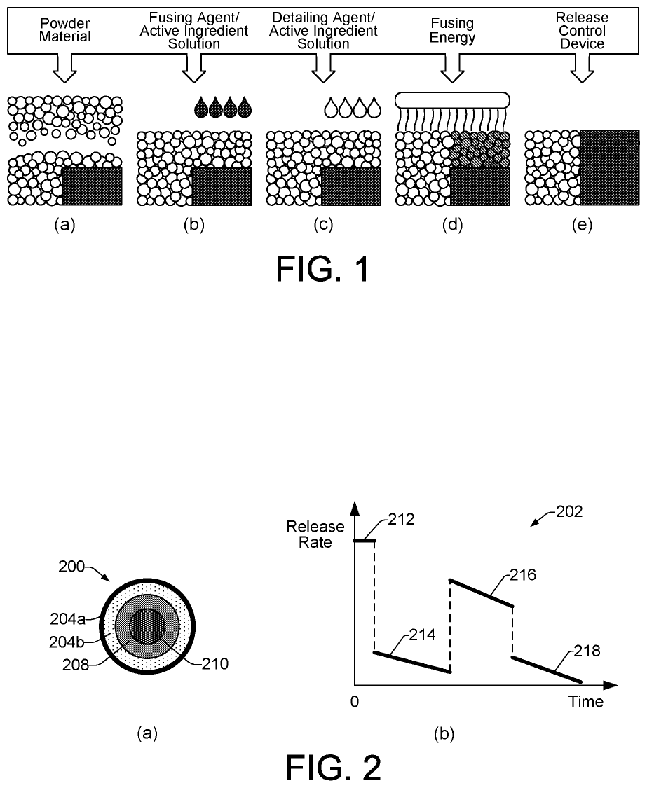

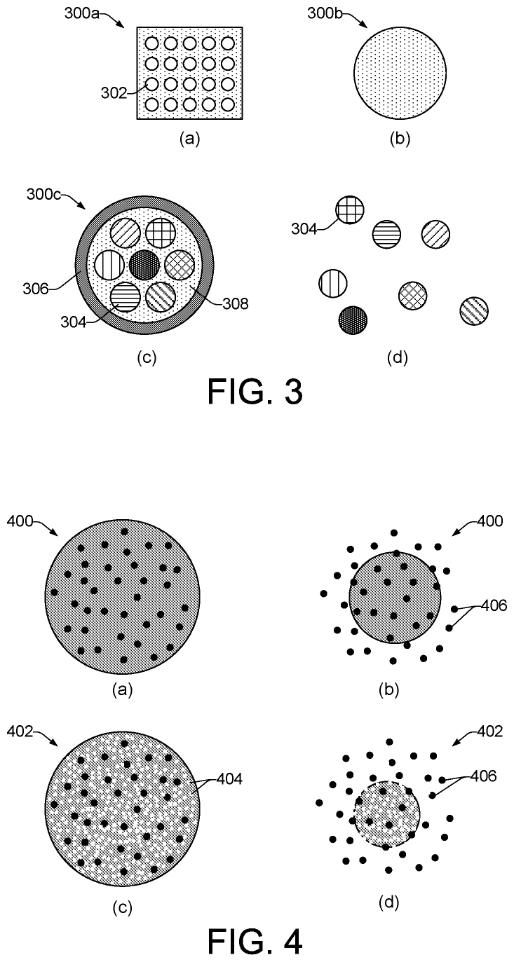

FIG. 1 shows a cross-sectional view of an example 3D printing process in which erosion-controlled release devices can be produced for erosion-controlled release of active ingredients. In the example process, erosion-controlled release devices can be produced layer-by-layer, through the selective deposition of liquid solutions and the application of fusing energy onto successive layers of powder material. FIG. 2 shows a cross-sectional view of an example erosion-controlled release device formed in an example 3D printing process. In the FIG. 2 example, the release profiles of multiple active ingredients are graphically illustrated to demonstrate erosion-controlled release based on the geometry of the erosion-controlled release device. FIG. 3 shows cross-sectional views of additional examples of erosion-controlled release devices in which the release of active ingredients can be controlled according to the geometry of the device.

Referring now generally to FIG. 1, in an example 3D printing process, a layer of powder material can be applied across a work space of a 3D printing device as shown in FIG. 1a. The work space can comprise, for example, the build platform of the device. The powder material can be applied over a previously applied powder layer (as shown in FIG. 1a), or directly onto the build platform of the work space when it is a first layer. The powder material can comprise a variety of inactive materials such as biocompatible materials that are ingestible and/or implantable materials, including for example, polymers, organics, gelatin, polysaccharides, carrageenans, starch, cellulose, flour, and combinations thereof. Some organic materials such as starch and flour can be fused when mixed with polymers due to the fusion of the polymers. In some examples, implantable materials can include metal and ceramic compositions in the powder material. Thus, the powder material can comprise an inactive substance that serves as an excipient carrier material to transport and deliver an active ingredient when ingested or implanted, for example. In some examples, the powder material can also comprise an active ingredient. Thus, the powder material may comprise a homogeneous mixture of inactive biocompatible powder material and an active ingredient in a powder form. Alternatively, or additionally, the powder material itself may be composed of an inactive biocompatible powder material and an active ingredient, such that each particle of the powder material consists of some ratio of inactive to active substance. In such examples where an active ingredient is included in the mixture or composition of the powder material, the liquid solution deposited onto the powder material (e.g., shown in FIG. 1b) may not include any active ingredient. That is, the liquid solution deposited onto the powder material may just be a liquid fusing agent.

As shown in FIG. 1b, a liquid solution can be selectively applied onto the powder layer where the particles of powder material are to be fused or sintered together. The liquid solution can comprise a fusing agent, an active ingredient, and/or a mixture of fusing agent and an active ingredient. In some examples, a fusing agent can be applied to the powder material separately, with or without a separate application of an active ingredient. In some examples, an active ingredient can be applied to the powder material separately, with or without a separate application of a fusing agent. In some examples, where an active ingredient is applied separately, without a fusing agent, the active ingredient can function both as an active ingredient and as a fusing agent, such as when an active ingredient is IR absorptive. A liquid solution comprising a mixture of one or multiple active ingredients (AI) as solute within a fusing agent (FA) as the solvent, may be referred to alternately herein as an “FA-AI solution”.

As shown in FIG. 1c, a liquid solution comprising a detailing agent can be selectively applied onto the powder layer where fusing of the powder material is to be reduced, prevented, or otherwise inhibited or altered. Detailing agents can include cooling agents and defusing agents, as discussed below. In some examples, a liquid solution comprising a mixture of detailing agent and an active ingredient can be selectively applied onto the powder layer. A liquid solution comprising a mixture of one or multiple active ingredients (AI) as solute within a detailing agent (DA) as the solvent, may be referred to alternately herein as a “DA-AI solution”. In general, the terms “fusing”, “fuse”, “fused”, and the like, indicate heating particles of the powder material to a level that involves fulling melting the particles to achieve solidification of the particles as a homogeneous part. The terms “sintering”, “sinter”, “sintered”, and the like, indicate heating particles of the powder material to a level that does not involve fulling melting the particles, but instead involves heating the particles of powder material to the point that the powder can fuse together on a molecular level. Thus, sintering generally enables control over the porosity of the material. However, because sintering involves a level of fusing particles together, the terms “fusing”, “fuse”, “fused”, may at times be used interchangeably with the terms “sintering”, “sinter”, “sintered”, depending on the context of the description. Thus, depending on the description, “fusing” may be used to indicate the solidification of particles of powder material that have not actually been fully melted, but instead have been partially melted. For example, in some instances a detailing agent can be deposited to reduce the fusing of particles within a particular area of powdered material in order to create porosity. In another example, an amount of fusing energy can be controlled (e.g., reduced) to a degree that particles of powdered material are partially melted rather than fully melted. Such actions may alternately be described as sintering, partial fusing, reduced fusing, and so on.

Referring generally to FIGS. 1b and 1c, fusing agents can comprise, for example, colored liquids such as carbon black ink that effectively target fusing energy (e.g., from an infrared light source) onto specific areas of the powder layer. Fusing agents can include water-based dispersions comprising a radiation absorbing agent such as an infrared light absorber, a near infrared light absorber, or a visible light absorber. Dye based and pigment based colored inks are examples of inks that include visible light absorbing agent. Darker fusing agents applied to the powder material generally cause a greater absorption of fusing energy into the powder, which causes higher temperatures and an increased melting and fusing together of the particles of powder. Detailing agents can comprise cooling agents and defusing agents. Detailing agents that comprise cooling agents can comprise, for example, liquids such as water that can cool the powder material during the application of fusing energy to prevent the powder from fully melting or fusing through controlling temperature. Detailing agents that comprise defusing agents can inhibit fusing chemically or mechanically. Such detailing agents can include other liquids such as silicon or oil that can be applied to mechanically and/or chemically inhibit fusing or sintering of the powder material.

As noted above, an active ingredient can include any of a variety of active pharmaceutical ingredients, drugs, medications, nutrients, and/or other ingredients to be consumed or applied for the treatment of various medical, nutritional, and/or other health related conditions. As shown in FIG. 1b, a liquid FA-AI solution comprising a mixture of one or multiple active ingredients within a fusing agent can be applied to the powder layer, for example, by jetting droplets of the liquid FA-AI solution through an inkjet printhead. Jetting the FA-AI solution enables precise placement of the fusing agent and the active ingredients onto the powder layer. The concentration of active ingredients within the FA-AI solution can be adjusted as one way to control dosing of the active ingredient. Likewise, a liquid DA-AI solution of detailing agent and active ingredients is also jettable to enable precise placement of the detailing agent and active ingredients onto the powder layer.

The selective application of fusing agents, active ingredients, FA-AI solutions, detailing agents, cooling agents, DA-AI solutions, and other like liquids to each powder layer, along with the subsequent application of fusing energy, enables a layer-by-layer formation of the surface boundary of an erosion-controlled release device, as well as the formation of the internal structure of the device. Thus, as each layer is fused, the boundary of the erosion-controlled release device can take on a particular geometric shape, while the internal structure of the device can take on particular characteristics. Structural characteristics of an erosion-controlled release device can be controlled through the selective application of fusing agents, detailing agents, and fusing energy (as discussed below). For example, the selective application of fusing agent and/or detailing agents enables the device to take on a variety of different structural characteristics, such as different porosities throughout the device, different levels of free or unfused powder material within the boundary of the device, and so on.

As shown in FIG. 1d, fusing energy can be applied to the powder layer after fusing agents, active ingredients, FA-AI solutions, detailing agents and DA-AI solutions have been applied. Fusing energy can be applied in a variety of ways, including for example, as infra-red (IR) radiation, near IR radiation, UV light, visible light emitting diodes (LEDs), lasers with specific wavelengths, heat lamps, and so on. Fusing energy can be controllably applied to each powder layer to control the level of fusing and/or sintering of the powder material. As noted above with regard to FIGS. 1b and 1c, the level of fusing and/or sintering of the powder material also depends on the fusing agents and detailing agents that may have been applied to the powder. Control over the level of fusing enables the creation of erosion-controlled release devices with varying structural characteristics. Such characteristics can include, for example, the surface geometry of the erosion-controlled release device, the internal porosity of the erosion-controlled release device, the amount of unfused powder material that remains free within the erosion-controlled release device, and so on. Higher fusing energies, prolonged fusing exposure, and multiple fusing exposures, can increase the melting of powder material and thereby decrease the porosity of an erosion-controlled release device. Lower fusing energies, reduced fusing exposure, and fewer fusing exposures, can reduce the melting of powder material and thereby increase the porosity of the device. The amount of melting or sintering of powder material additionally depends on the amount and types of fusing and detailing agents that may be applied to the powder.

FIG. 1e shows an example of a portion of a layer of fused powder material, such as a layer of an erosion-controlled release device. As noted above, FIG. 2 shows a cross-sectional view of an example erosion-controlled release device 200 (FIG. 2a) that can be formed in an example 3D printing process, in addition to a graphical illustration 202 (FIG. 2b) of the erosion-controlled release of multiple active ingredients according to the geometric structure of the device 200. FIG. 3 shows cross-sectional views of additional examples of erosion-controlled release devices 300 (illustrated as devices 300a, 300b, 300c) in which the release of active ingredients can be controlled according to the geometry of the device. The erosion-controlled release devices 200 and 300, comprise active ingredient release profiles for releasing an active ingredient, or multiple active ingredients, that are controlled according to the geometry of the device. Referring to FIG. 2a, for example, a multi-drug erosion-controlled release device 200 has been produced with a release profile that is controlled by the relative geometric locations of the different active ingredients (drugs) within the device 200. More specifically, the active ingredients 204, 206, 208, 210, have been arranged in an order from the outside boundary of the device 200 to the inside of the device. Thus, the release of active ingredients 204, 206, 208, 210, can occur in order, from the outside to the inside of the device 200 as the device erodes or dissolves.

This erosion-controlled release profile of the multiple active ingredients of device 200 can be shown in graph 202 of FIG. 2b. Erosion of the device 200 occurs at the outer surface and proceeds inward. Thus, the release of active ingredient 204a from device 200 is shown at line segment 212 of graph 202 as the outer boundary or surface erodes first. In this example, the outer boundary of device 200 comprises a high density of active ingredient 204a and a high porosity that provides for a “burst release” of the active ingredient 204a as the outer surface boundary of the device quickly erodes. The line segment 212 of FIG. 2b shows this high release rate that is a constant release rate over a short time duration as the thin outer boundary of the device 200 erodes quickly. In this example, active ingredients 204a and 204b can comprise the same active ingredient, but in different concentrations. That is, active ingredient 204a can be deposited at the geometric boundary of the device 200 in higher concentrations than the same active ingredient 204b is deposited just inside the geometric boundary of the device 200. Thus, the line segment 212 shows a high release rate of active ingredient 204a that is a constant release rate over a short time duration, followed by the line segment 214 which represents the subsequent release of active ingredient 204b. As the erosion of device 200 proceeds from the outside boundary inward, the release rate drops as shown by line segment 214, due to the low ink density, low concentration of active ingredient 204b, and low porosity. The low density and low porosity cause the slow release rate. Furthermore, the line segment 214 shows a decreasing release rate over time as the remaining outer surface area of device 200 continues to erode from the outside inward. Once the device 200 has eroded through the area of the active ingredient 204b, the release rate of the device 200 jumps up as shown at line segment 216, due to the increased concentration of the active ingredient 208 and increased porosity of the device. The release rate of the active ingredient 208 then continues to decrease as shown at line segment 216, due to the continuing erosion of device 200 from the outside boundary inward. Once the erosion of device 200 has proceeded through the area of the active ingredient 208, the release rate of the device 200 drops down due to the decreased concentration of the final active ingredient 210, as shown at line segment 218. The release rate of the final active ingredient 210 then continues to decrease as shown at line segment 218, due to the continuing erosion of device 200 from the outside boundary inward.

Referring still to the erosion-controlled release device 200 of FIG. 2a, the corresponding release profile shown in FIG. 2b can be achieved through an example 3D printing process. Active ingredients can be applied to the device 200 from the outside boundary toward the inside of the device. For example, for a given powder layer of the device 200, a first FA-AI solution comprising a first active ingredient 204a can be applied (i.e., jetted) onto the illustrated layer at the outer boundary of the device 200. On the same illustrated powder layer of the delivery device 200, a second FA-AI solution comprising a second active ingredient 204b can be applied within, or inside, the area of the first active ingredient 204a. In some examples, a second active ingredient 204b can comprise a different type of active ingredient, or it may comprise a different concentration of the same type of active ingredient as the first active ingredient 204a. As shown in FIG. 2a, a third FA-AI solution comprising a third active ingredient 208 can be applied within the area of the active ingredient 206, progressing toward the center of the device 200. A final FA-AI solution comprising a fourth active ingredient 210 can then be applied in the center area of the illustrated layer of the device 200.

Additional or fewer active ingredients can be included in an erosion-controlled release device by this example 3D printing process. Furthermore, in some examples, a detailing agent solution may be applied to the illustrated layer of the delivery device 200 instead of or in addition to the active ingredients 204a, 204b, 208, 210. A fusing energy applied to the illustrated layer of device 200 can then be controlled to fuse the areas 204a, 204b, 208, 210, according to various factors including the intensity of the fusing energy, the time of exposure to the fusing energy, the number of exposures to the fusing energy, the types and amounts of fusing agent and detailing agent applied, and so on. The fusing can control the porosity, for example, of each area 204a, 204b, 208, 210, of the device 200. In some examples, the fusing can control the nature of the active ingredients stored or trapped within the areas 204a, 204b, 208, 210, of the device 200. For example, in some instances, greater or lesser fusing can be applied to control the state of the active ingredients. In different examples, active ingredients within an erosion-controlled release device 200 can comprise solids, liquids, gases, solid-liquid combinations, solid-gas combinations, solid-liquid-gas combinations, and so on.

Referring now to FIG. 3, additional examples of erosion-controlled release devices 300 (illustrated as devices 300a, 300b, 300c) are shown in which the release of active ingredients can be controlled according to the geometry of the devices. Referring to FIG. 3a, a layer or cross-section of an example erosion-controlled release device 300a formed as a matrix structure is shown. Such a matrix structure provides a larger surface area for greater exposure of an active ingredient 302, which can result in a fast release profile. During an example 3D printing process, such a structure can comprise a homogeneous amount of active ingredient 302 filling the matrix structure. A homogeneous distribution of active ingredient can provide a release profile in which the rate of release of the active ingredient decreases with time, through erosion of the device 300a. In some examples, an increasing amount of active ingredient 302 can fill the matrix, from the outside of the device to the inside of the device. An increasing distribution of active ingredient can provide a release profile in which the rate of release remains constant or increases.

Referring to FIG. 3b, a layer or cross-section of an example erosion-controlled release device 300b is shown in which the device 300b provides a reduced surface area. The release profile of such an erosion-controlled release device 300b can be slower than that of a matrix structure or other structure.

FIG. 3c shows a cross-sectional view of an example erosion-controlled release device 300c that comprises a system of mini-tablets 304 formed in an example 3D printing process. An example delivery device 300c can provide multiple modified release profiles for different active ingredients associated with each mini-tablet 304. Different release profiles can include, for example, extended release, delayed release, pulsed release, binary release, and so on. In some examples, each mini-tablet 304 can comprise a distinct active ingredient. In some examples, during an example 3D printing process, the level of fusing applied to each mini-tablet 304, and thus the structure of each mini-tablet 304, can vary based on the types and amounts of applied fusing agents, detailing agents, and fusing energy.

Referring to FIG. 3c, during an example 3D printing process, a liquid fusing agent 306 without an active ingredient, can be applied onto a layer or cross-section of an example erosion-controlled release device 300c. Upon fusing, the fusing agent area 306 can provide the boundary or outer surface of the erosion-controlled release device 300c. Numerous different solutions of fusing agent and active ingredient (i.e., FA-AI solutions) can be deposited/jetted within the boundary area 306, and then fused with fusing energy to form the different mini-tablets 304. In some examples, the interior structure of the device 300c can comprise unfused or partially fused powder material 308 on which a detailing agent has been deposited. The erosion-controlled release device 300c can release the mini-tablets 304 as shown in FIG. 3d, after which the unique release profiles of each mini-tablet 304 can control the release of a unique active ingredient.

FIG. 4 shows examples of erosion-controlled release devices exhibiting different erosion, or dissolution, schemes. In FIG. 4a, an erosion-controlled release device 400 (e.g., a tablet) is shown prior to being eroded. In FIG. 4b, the device 400 is in the process of eroding. In FIG. 4c, a different erosion-controlled release device 402 is shown prior to being eroded. In FIG. 4d, the device 402 is in the process or erosion. The device 400 has little or no porosity, while the device 402 has an amount of porosity as indicated by the white, empty voids 404 evident in both FIGS. 4c and 4d. When comparing the progress of the erosion process between the partially eroded devices of FIGS. 4b and 4d, it is apparent that the erosion-controlled release device 402 in FIG. 4d erodes more quickly than the erosion-controlled release device 400 in FIG. 4b. Furthermore, the more porous device 402 of FIG. 4d releases its active ingredients 406 faster than the less porous device 400 of FIG. 4b. Thus, erosion-controlled release devices that have greater amounts of porosity can dissolve more quickly and have a faster active ingredient release rate than similar erosion-controlled release devices having less porosity. This is so, because devices with little or no porosity have limited surface area (i.e., the outer surface of the device) that comes in contact with water or other digestive fluids, while devices with greater porosity have greater surface area (i.e., the outer surface and the porous inner surface) that comes in contact with water or other digestive fluids, which results in faster dissolution and faster release rates.

Example 3D printing processes described herein comprise fusing operations that enable accurate control over the porosity of erosion-controlled release devices through the control of various fusing related factors. Such fusing factors can include, for example, the amount of fusing energy applied to and absorbed by layers of powder material, the intensity or power of the fusing energy applied, the number of fusing applications or passes used, the duration of fusing applications, the types of fusing agents applied to the powder material, the types of detailing agents applied to the powder material, and so on. In general, higher levels of porosity are achieved with less fusing, such as when sintering occurs. Conversely, lower levels of porosity are achieved with increased fusing, such as when fusing causes the powder material to fully melt and fully fuse together. In some examples, free powder material that has experienced no fusing can have on the order of 50% porosity, while partially fused powder (i.e., sintered powder) can have on the order of 10% porosity, and fully fused powder that has been fully melted can have 0% porosity. Accordingly, the use of fusing in example 3D printing processes described herein to accurately control the porosity of erosion-controlled release devices enables control over the release profiles of active ingredients.

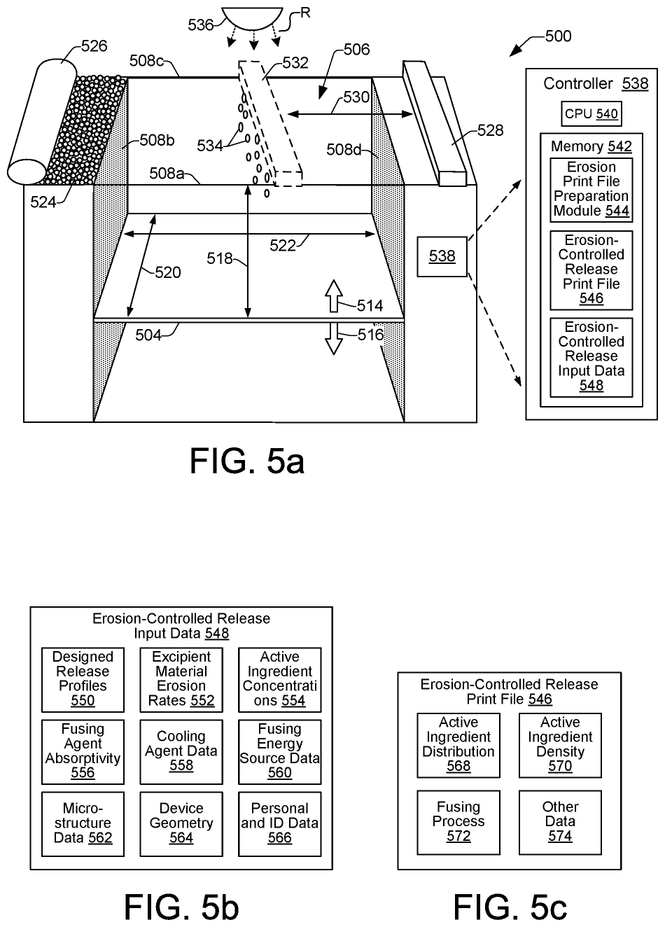

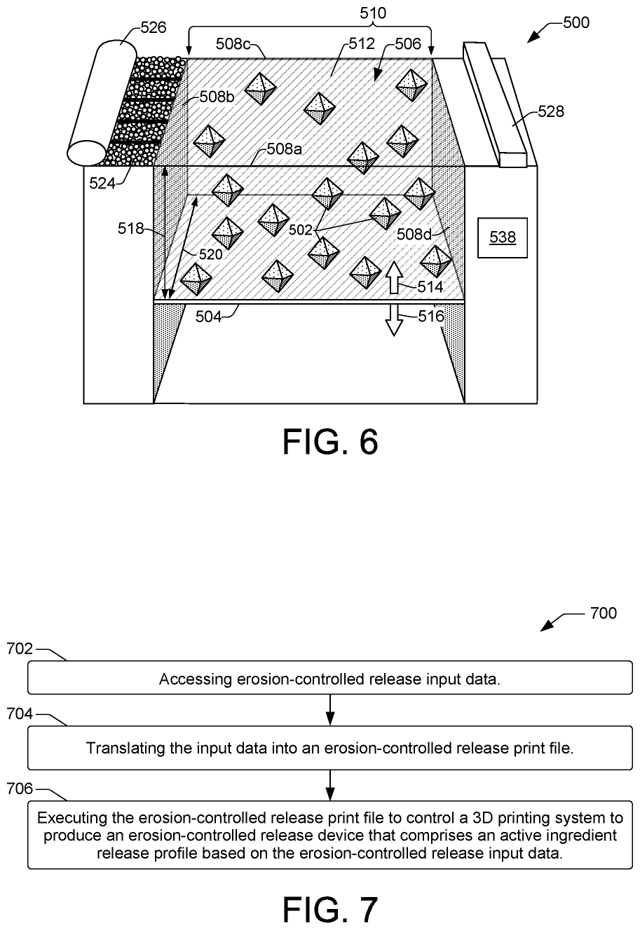

FIG. 5a shows a perspective view of an example of a 3D printing system 500 suitable for printing erosion-controlled release devices 502 (FIG. 6; e.g., tablets, pills, implants) for controlled release of active ingredients according to examples described herein. FIG. 6 shows a perspective view of an example of the 3D printing system 500 in which example erosion-controlled release devices 502 have been printed. Referring to FIGS. 5a and 6, the 3D printing system 500 includes a moveable printing platform 504, or build platform 504. The printing platform 504 can serve as the floor to a work space 506 in which erosion-controlled release devices 502 can be printed. The work space 506 can include fixed walls 508 (illustrated as front wall 508a, side wall 508b, back wall 508c, side wall 508d) that border the printing platform 504. The fixed walls 508 can contain a build volume 510 (FIG. 2a) comprising powdered build material that is deposited into the work space 506 during printing of an erosion-controlled release device 502. During printing, the build volume 510 can include all or part of one or a number of erosion-controlled release devices 502 either completed or partially completed in which powder layers have had fusing agents, active ingredients (e.g., FA-AI solutions), and fusing energy applied. The build volume 510 can also include non-processed powder material that surrounds the completed or partially completed erosion-controlled release devices 502. Non-processed powder material can comprise a volume of reclaimable powder material 512 (illustrated in FIG. 6 as lightly shaded lines). For purposes of this discussion and to help illustrate different elements and functions of the 3D printing system 500, the front wall 508a of the work space 506 is shown as being transparent.

The printing platform 504 is moveable within the work space 506 in an upward and downward direction as indicated by up arrow 514 and down arrow 516, respectively. When the printing of erosion-controlled release devices 502 begins, the printing platform 504 can be located in an upward position toward the top of the work space 506 as a first layer of powdered material is deposited onto the printing platform 504 and processed, for example, by applying fusing agents, detailing agents, active ingredients, and fusing energy. After a first layer of powder material has been processed, the printing platform 504 can move in a downward direction 516 as additional layers of powdered material are deposited onto the platform 504 and processed. Thus, the printing platform 504 can increase the height 518 dimension of the work space 506 to accommodate the production of additional erosion-controlled release devices 502 by continuing to move downward 516. While the height 518 of the work space 506 is adjustable by movement of the printing platform 504 in a vertical direction, the depth 520 and width 522 dimensions of the work space 506 are fixed by the horizontal dimensions of the platform within the fixed walls 508.

Referring still to FIGS. 5a and 6, the example 3D printing system 500 includes a supply 524 of powdered material, or powder. As noted above, the powder material, alternately referred to herein as “powder”, can comprise inactive biocompatible powder materials such as polymers, organics, gelatin, polysaccharides, carrageenans, starch, cellulose, flour, and combinations thereof, that comprise ingestible and/or implantable materials. In some examples, the powder material in a powder supply 524 can also comprise an active ingredient. In some examples, a 3D printing system 500 can comprise multiple powder supplies 524 that contain different types of powder materials, and/or different mixtures of inactive biocompatible powder material and active ingredients. Powder materials from a supply 524 serve as the material of the erosion-controlled release devices 502 which comprise active ingredient excipients-carriers. The 3D printing system 500 can feed powder material from a supply 524 into the work space 506 using a powder spreader 526 to controllably spread the powder into layers over the printing platform 504, and/or over other previously deposited layers of powder. In different examples, a powder spreader 526 can include a roller, a blade, or another type of material spreading device.

The example 3D printing system 500 also includes a liquid solution dispenser 528. While other types of liquid solution dispensers are possible, the example dispenser 528 shown and described herein comprises a printhead 528 or printheads, such as thermal inkjet or piezoelectric inkjet printheads. The example printhead 528 comprises a drop-on-demand printhead having an array of liquid ejection nozzles suitable to selectively deliver liquid fusing agents, liquid active ingredients, liquid detailing agents, solutions of fusing agent and active ingredient (i.e., FA-AI solutions), or other liquids, onto a layer of powder that has been spread onto the printing platform 504. In some examples, the printhead 528 has a length dimension that enables it to span the depth 520 of the work space 506 in a page-wide array arrangement as it scans over the work space 506 to apply droplets of FA-AI solution onto layers of powder within the work space 506. In FIG. 5a, an example scanning motion 530 of the printhead 528 (shown by dashed-line printhead representation 532) is illustrated by direction arrow 530 as the printhead 528 scans across the work space 506 and ejects droplets of FA-AI solution 534 into the work space 506. Although not shown in the example of FIG. 5a, in an actual printing scenario portions of erosion-controlled release devices 502 would be present within the work space 506 as the printhead 528 scans over the work space and ejects droplets of the FA-AI solution 534, such as the erosion-controlled release devices 502 shown in FIG. 6.

As shown in FIG. 5a, the example 3D printing system 500 also includes a fusing energy source such as radiation source 536 (not shown in FIG. 6). The radiation source 536 can apply radiation R to layers of powder material in the work space 506 to facilitate the heating and fusing of the powder. In some examples, a fusing agent or FA-AI solution 534 can be selectively applied by printhead 528 to a layer of powder material to enhance the absorption of the radiation R and convert the absorbed radiation into thermal energy, which can elevate the temperature of the powder sufficiently to cause curing (e.g., fusing, melting, sintering) of the particles of the powder. The radiation source 536 can be implemented in a variety of ways including, for example, as a curing lamp or as light emitting diodes (LEDs) to emit IR, near-IR, UV, or visible light, or as lasers with specific wavelengths. The radiation source 536 can depend in part on the type of fusing agent and/or powder being used in the printing process. The radiation source 536 can be attached to a carriage (not shown) and can be stationary or scanned across the work space 506.

The example 3D printing system 500 additionally includes an example controller 538. The controller 538 can control various operations of the printing system 500 to facilitate the printing of erosion-controlled release devices 502 as generally described above. Such operations can include, for example, workflow operations for preparing erosion-controlled release 3D print files, as well as operations to execute such print files to produce erosion-controlled release devices with particular active ingredient release profiles.

As shown in FIG. 5a, an example controller 538 can include a processor (CPU) 540 and a memory 542. The controller 538 may additionally include other electronics (not shown) for communicating with and controlling various components of the 3D printing system 500. Such other electronics can include, for example, discrete electronic components and/or an ASIC (application specific integrated circuit). Memory 542 can include both volatile (i.e., RAM) and nonvolatile memory components (e.g., ROM, hard disk, optical disc, CD-ROM, flash memory, etc.). The components of memory 542 comprise non-transitory, machine-readable (e.g., computer/processor-readable) media that can provide for the storage of machine-readable, executable, coded program instructions, data structures, program instruction modules, JDF (job definition format), 3D Manufacturing Format (3MF) print files, and other data and/or instructions executable by a processor 540 of the 3D printing system 500.

An example of executable instructions to be stored in memory 542 can include instructions associated with an erosion print file preparation module 544, and an erosion-controlled release print file 546. An example of stored data can include erosion-controlled release input data 548. Instructions from the erosion print file preparation module 544 can be executable to access various input data from the erosion-controlled release input data 548 and to prepare an erosion-controlled release print file 546 using the erosion-controlled release input data 548. Instructions and commands from an erosion-controlled release print file 546 can be executable by controller 538 to control components and functions of 3D printing system 500 to produce an erosion-controlled release device 502. The erosion-controlled release device 502 can have a particular erosion-controlled release profile that is consistent with the erosion-controlled release input data 548.

FIG. 5b shows an example of data components within the erosion-controlled release input data 548 that can be used to prepare an erosion-controlled release print file 546. In some examples, a 3D printing system 500 can receive data components from a data source (not shown), such as a network or a computer coupled to the 3D printing system 500. In some examples, such as when data components may be missing from the erosion-controlled release input data 548, a 3D printing system 500 can retrieve data components from a data source (not shown), such as a network or a computer coupled to the 3D printing system 500. FIG. 5c shows an example of print file components within an erosion-controlled release print file 546 that can be implemented and/or executed by controller 538 to cause the 3D printing system 500 to produce an erosion-controlled release device 502.

As shown in FIG. 5b, erosion-controlled release input data 548 can include designed release profiles 550 for active ingredients that are to be included in an erosion-controlled release device 502. A designed release profile 550 can include information from a manufacturer of an active ingredient, for example, that indicates the optimum release timing and release dosing to achieve the most beneficial therapeutic effects from the active ingredient. Erosion-controlled release input data 548 can also include data on the excipient powder material erosion rates 552. This data can include, for example, material erosion rates according to different microstructures within an erosion environment. Input data 548 can also include data on the concentrations of active ingredients 554 within the liquid inks and/or liquid fusing agents to be deposited (i.e., jetted) onto the excipient powder material, data specifying the energy absorptivity 556 of the fusing agents, data specifying an amount of cooling caused by cooling agents 558, hardware specification data characterizing a fusing energy source 560 of the 3D printing system 500, data indicating which internal microstructures 562 are to be produced within an erosion-controlled release device, geometric data 564 indicating an external boundary and initial surface area of an erosion-controlled release device, personal data 566 about an intended user, identification data 566 to identify active ingredients within an erosion-controlled release device and the intended user of the erosion-controlled release device, and so on. Personal data 566 about an intended user can include, for example, a user's height, weight, age, sex, living environment, working environment, diet, exercise habits, and so on. and other data.

As shown in FIG. 5c, print file components within an erosion-controlled release print file 546 can include information, commands and instructions translated from data in the erosion-controlled release input data 548 that can be understood and executed by the controller 538 to produce an erosion-controlled release device in a 3D printing process. The print file components can include active ingredient distribution 568 commands and data that define locations for active ingredient droplets to be applied to different powder layers, active ingredient density 570 commands and data that define the density or amounts of active ingredient/ink droplets to be applied to different powder layers, fusing process data and commands 572 that can define fusing source intensity, fusing application durations, and the number of fusing exposures for different powder layers, and other data 574 such as text data to be included on the erosion-controlled release device to identify the active ingredient(s), the intended target user, and so on.

FIGS. 7, 8, and 9 are flow diagrams showing example methods 700, 800, and 900, of producing erosion-controlled release devices. Method 800 comprises extensions of method 700 that incorporate additional details. Methods 700, 800, and 900 are associated with examples discussed above with regard to FIGS. 1-6, and details of the operations shown in methods 700, 800, and 900 can be found in the related discussion of such examples. The operations of methods 700, 800, and 900 may be embodied as programming instructions stored on a non-transitory, machine-readable (e.g., computer/processor-readable) medium, such as memory 542 shown in FIG. 5a. In some examples, implementing the operations of methods 700, 800, and 900 can be achieved by a processor, such as a processor 540 of FIG. 5a, reading and executing the programming instructions stored in a memory 542. In some examples, implementing the operations of methods 700, 800, and 900 can be achieved using an ASIC and/or other hardware components alone or in combination with programming instructions executable by a processor 540.

The methods 700, 800, and 900 may include more than one implementation, and different implementations of methods 700, 800, and 900 may not employ every operation presented in the respective flow diagrams of FIGS. 7, 8, and 9. Therefore, while the operations of methods 700, 800, and 900 are presented in a particular order within their respective flow diagrams, the order of their presentations is not intended to be a limitation as to the order in which the operations may actually be implemented, or as to whether all of the operations may be implemented. For example, one implementation of method 800 might be achieved through the performance of a number of initial operations, without performing one or more subsequent operations, while another implementation of method 800 might be achieved through the performance of all of the operations.

Referring now to the flow diagram of FIG. 7, an example method 700 of producing erosion-controlled release devices begins at block 702 with accessing erosion-controlled release input data. The method 700 can continue with translating the input data into an erosion-controlled release print file, as shown at block 704. As shown at block 706, the method can include executing the erosion-controlled release print file to control a 3D printing system to produce an erosion-controlled release device that comprises an active ingredient release profile based on the erosion-controlled release input data.

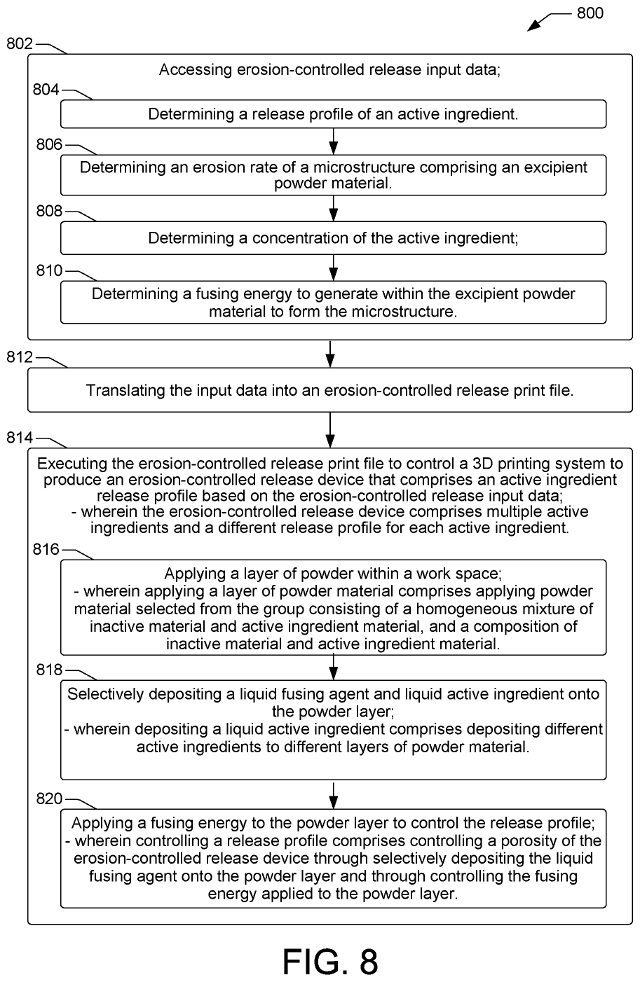

Referring now to the flow diagram of FIG. 8, another example method 800 of producing erosion-controlled release devices is shown. As noted above, method 800 comprises extensions of method 700 that incorporate additional details. Thus, method 800 can begin at block 802 with accessing erosion-controlled release input data. In different examples, accessing erosion-controlled release input data can include determining a release profile of an active ingredient, determining an erosion rate of a microstructure comprising an excipient powder material, determining a concentration of the active ingredient, and determining a fusing energy to generate within the excipient powder material to form the microstructure, as shown respectively at blocks 804, 806, 808, and 810. The method 800 can continue at block 812 with translating the input data into an erosion-controlled release print file. As shown at block 814, the erosion-controlled release print file can be executed to control a 3D printing system to produce an erosion-controlled release device that comprises an active ingredient release profile based on the erosion-controlled release input data. In some examples, the erosion-controlled release device comprises multiple active ingredients and a different release profile for each active ingredient.

In some examples, as show at block 816, executing the erosion-controlled release print file can include applying a layer of powder within a work space. In some examples, applying a layer of powder material comprises applying powder material selected from the group consisting of a homogeneous mixture of inactive material and active ingredient material, and a composition of inactive material and active ingredient material. In some examples, as show at block 818, executing the erosion-controlled release print file can include selectively depositing a liquid fusing agent and liquid active ingredient onto the powder layer. In some examples, depositing a liquid active ingredient comprises depositing different active ingredients to different layers of powder material. As show at block 820, executing the erosion-controlled release print file can include applying a fusing energy to the powder layer to control the release profile. In some examples, controlling a release profile comprises controlling a porosity of the erosion-controlled release device through selectively depositing the liquid fusing agent onto the powder layer and through controlling the fusing energy applied to the powder layer.

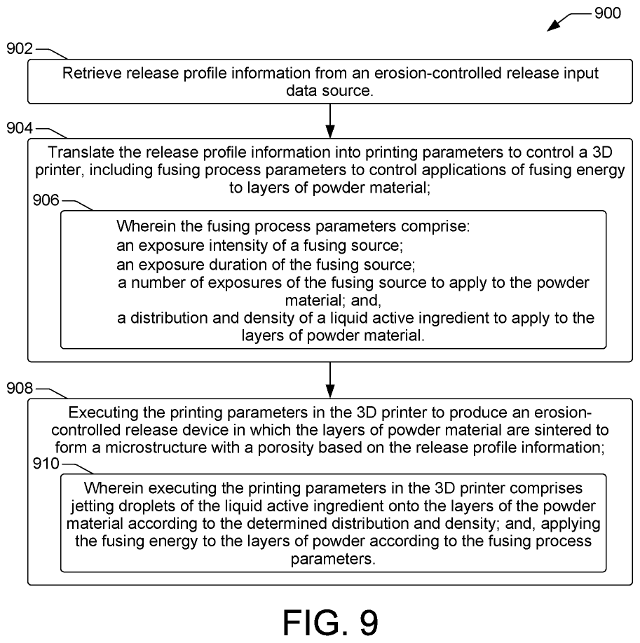

Referring now to the flow diagram of FIG. 9, another example method 900 of producing erosion-controlled release devices is shown. As shown at block 902, the method can include retrieving release profile information from an erosion-controlled release input data source. The method can also include translating the release profile information into printing parameters to control a 3D printer, including fusing process parameters to control applications of fusing energy to layers of powder material, as shown at block 904. In some examples, as shown at block 906, the fusing process parameters can comprise an exposure intensity of a fusing source, an exposure duration of the fusing source, a number of exposures of the fusing source to apply to the powder material, and a distribution and density of a liquid active ingredient to apply to the layers of powder material. Continuing at block 908, method 900 can further include executing the printing parameters in the 3D printer to produce an erosion-controlled release device in which the layers of powder material are sintered to form a microstructure with a porosity based on the release profile information. In some examples, as shown at block 910, executing the printing parameters in the 3D printer can include jetting droplets of the liquid active ingredient onto the layers of the powder material according to the determined distribution and density, and applying the fusing energy to the layers of powder according to the fusing process parameters.

What is claimed is:

1. A method of producing an erosion-controlled release device, comprising:
accessing erosion-controlled release input data;
translating the input data into an erosion-controlled release print file; and,
executing the erosion-controlled release print file to control a 3D printing system to produce an erosion-controlled release device that comprises an active ingredient release profile based on the erosion-controlled release input data.

2. A method as in claim 1, wherein executing the erosion-controlled release print file comprises:
applying a layer of powder within a work space;
selectively depositing a liquid fusing agent and liquid active ingredient onto the powder layer; and,
applying a fusing energy to the powder layer to control the release profile.

3. A method as in claim 2, wherein controlling a release profile comprises controlling a porosity of the erosion-controlled release device through selectively depositing the liquid fusing agent onto the powder layer and through controlling the fusing energy applied to the powder layer.

4. A method as in claim 2, wherein applying a layer of powder material comprises applying powder material selected from the group consisting of a homogeneous mixture of inactive material and active ingredient material, and a composition of inactive material and active ingredient material.

5. A method as in claim 1, wherein the erosion-controlled release device comprises multiple active ingredients and a different release profile for each active ingredient.

6. A method as in claim 2, wherein depositing a liquid active ingredient comprises depositing different active ingredients to different layers of powder material.

7. A method as in claim 1, wherein accessing erosion-controlled release input data comprises;
determining a release profile of an active ingredient;
determining an erosion rate of a microstructure comprising an excipient powder material;
determining a concentration of the active ingredient; and,
determining a fusing energy to generate within the excipient powder material to form the microstructure.

8. A 3D printing system for producing erosion-controlled release devices comprising:
a memory device comprising erosion-controlled release input data; and,
a processor programmed with instructions from a print file preparation module to prepare an erosion-controlled release print file based on the erosion-controlled release input data, the print file comprising commands to control the 3D printing system to produce an erosion-controlled release device.

9. A 3D printing system as in claim 8, wherein the erosion-controlled release print file comprises commands to control a fusing process for fusing layers of excipient powder material of the erosion-controlled release device.

10. A 3D printing system as in claim 8, wherein the erosion-controlled release input data comprises data selected from the group consisting of designed release profiles of active ingredients, excipient powder material erosion rates, active ingredient concentrations in liquids to be deposited onto excipient powder material, energy absorptivity of fusing agents, cooling effects of cooling agents, hardware specification data characterizing a fusing energy source, internal microstructure data, geometric data of the erosion-controlled release device, personal data of an intended user of the erosion-controlled release device, identification data to identify active ingredients within the erosion-controlled release device, and combinations thereof.

11. A 3D printing system as in claim 8, further comprising:
a printing platform on which to spread powder material from a powder supply into powder layers;
a liquid dispenser to selectively jet a liquid fusing agent and a liquid active ingredient onto the powder layers; and,
a fusing energy source to apply a fusing energy to the powder layers to produce a porosity within the erosion-controlled release device that achieves a release profile according to the erosion-controlled release input data.

12. A non-transitory machine-readable storage medium storing instructions that when executed by a processor of a three-dimensional (3D) printer for producing an erosion-controlled release device, cause the 3D printer to:
retrieve release profile information from an erosion-controlled release input data source;
translate the release profile information into printing parameters to control the 3D printer, including fusing process parameters to control applications of fusing energy to layers of powder material;
executing the printing parameters in the 3D printer to produce an erosion-controlled release device in which the layers of powder material are sintered to form a microstructure with a porosity based on the release profile information.

13. A medium as in claim 12, wherein the fusing process parameters comprise:
an exposure intensity of a fusing source;
an exposure duration of the fusing source; and,
a number of exposures of the fusing source to apply to the powder material.

14. A medium as in claim 13, wherein the fusing process parameters further comprise:
a distribution and density of a liquid active ingredient to apply to the layers of powder material.

15. A medium as in claim 14, wherein executing the printing parameters in the 3D printer comprises:
jetting droplets of the liquid active ingredient onto the layers of the powder material according to the determined distribution and density; and,
applying the fusing energy to the layers of powder according to the fusing process parameters.
